(19) United States
(12) Patent Application Publication
(10) Pub. No.: US 2003/0164168 A1
(43) Pub. Date: Sep. 4, 2003

(54) Bronchiopulmonary occulsion devices and lung volume reduction methods

(76) Inventor: David Peter Shaw, Christchurch (NZ)

Correspondence Address: Stephanie Seidman, Heller Ehrman White & McAuliffe, 7th Floor, 4350 La Jolla Village Drive, San Diego, CA, 92122-1246, US

(21) Appl. No.: 10/275,995

(22) PCT Filed: May 18, 2001
(86) PCT No.: PCT/NZ01/00092

(30) Foreign Application Priority Data
May 18, 2000 (NZ) 504621

(51) Int. Cl. A61M 16/00

(52) U.S. Cl. 128/200.24

(57) ABSTRACT

Lung volume reduction is performed by the placement of a device (2) into a branch of the airway (34) to prevent air from entering that portion of lung. This will result in adsorption atelectasis of the distal portion of lung. The physiological response in this portion of lung is hypoxic vaso-constriction. The net effect is for a portion of lung to be functionally removed, i.e. a selected portion of lung is removed from both the circulation and ventilation. The build up of secretions is accommodated by using a valve (5, 15, 29) in the obstructive device, the valve opening upon coughing etc.

### TECHNICAL FIELD

The present invention relates to devices for bronchiopulmonary occlusion, inter alia for inducing lung volume reduction, and surgical procedures using such devices, including methods of lung volume reduction.

### BACKGROUND ART

Emphysematous lungs are characterised by abnormally large air spaces. Lung compliance characteristics are such that the lung is ‘too large’ for its pleural cavity.

Lung volume reduction surgery (LVRS) was developed as an intervention procedure to alleviate respiratory distress in a patient with a minimal reserve. In this procedure, a portion of less efficient lung is removed under general anaesthetic, allowing the remaining lung to expand. The net effect is, paradoxically to improve respiratory function by excising a section of lung. LVRS is associated with moderate mortality, approximately 5% and frequently high morbidity such as prolonged air leakage. To optimiise patient outcome, selection criteria are strict and an extensive pre- and postoperative physiotherapy programme is undertaken. The length of hospitalisation for the surgery and initial postoperative care can be in the order of threemonths. The intervention, as a whole, is a very expensive procedure and generally is7 not covered by insurance schemes. In the USA, this high cost has resulted in the procedure being substantially funded within FDA approved trials.

Other indications for which the devices and methods of the present invention may be applied include bronchial occlusion for the treatment of spontaneous pneumothorax, persistent pneumothoraces and as an adjuvant to the chemotherapeutic treatment of tuberculosis.

### DISCLOSURE OF INVENTION

It is an object of the present invention to provide a relatively non-invasive and comparatively inexpensive lung volume reduction procedure by forming a temporary or permanent obstruction in a bronchus. It is a further object of the present invention to provide an efficient and relatively inexpensive occluding device which can be deployed by an endoscope. Endoscopic insertion of an obstructive device is likely to reduce mortality and morbidity compared with traditional surgery in patients having limited reserve and thus permit more liberal case selection.

The target site may be a portion of a trachobronchial tree. More preferably, the target site is a third or fourth generation bronchus. Preferably the occluding device is removable by endoscopic probe deployment and retrieval. If necessary, the occluding device can be compressed or deformed by the probe to facilitate removal. Optionally the device is biodegradable being composed of biocompatible material having a predetermined life span to provide temporary occlusion.

The blocking mechanism may be a transverse partitioning member such as an end wall, or resilient diaphragm. Alternatively, the blocking mechanism is an occlusive plug such as an inflatable balloon or pivotable stopper biased to a sealing position. Preferably, however, the blocking means is in the form of a one-way valve, which functions to allow the egress of gases or fluids from the targeted volume.

Lung volume reduction is thus performed by the placement of a device into a branch of the airway to prevent air from entering that portion of lung. This will result in adsorption atelectasis of the distal portion of lung. The physiological response in this portion of lung is hypoxic vasoconstriction. The net effect is for a portion of lung to be functionally removed, i.e. a selected portion of lung is removed from both the circulation and ventilation. The build up of secretions is accommodated by the valve in the obstructive device, the valve opening upon coughing etc.

### BRIEF DESCRIPTION OF THE DRAWINGS

By way of example only, preferred embodiments of the present invention are described in detail with reference to the accompanying drawings in which:

FIG. 1 is an end view of an occluding device in accordance with present invention;

FIG. 2 is a cross-sectional view of the occluding device taken on the line 2-2 in FIG. 1;

FIG. 3 shows in side elevation an occluding device according to a second embodiment of the invention;

FIG. 4 shows an end view of the device of FIG. 3;

FIG. 5 shows a delivery system for the device of FIG. 3;

FIG. 6 shows an inflation device;

FIG. 7 shows the device of FIG. 3 mounted on the inflation device;

FIG. 8 shows a further alternative embodiment of the occluding device;

FIG. 9 is an end view of a frame for an occluding device;

FIG. 10 is a side elevation of the frame of FIG. 9;

FIG. 11 is an end view of an occluding device incorporating the frame of FIGS. 9 and 10;

FIG. 12 is a cross-sectional elevation of the occluding device of FIG. 11, and

FIGS. 13 to 16 schematically illustrate methods of insertion and removal of the device of FIGS. 11 and 12.

### DESCRIPTION OF PREFERRED EMBODIMENTS

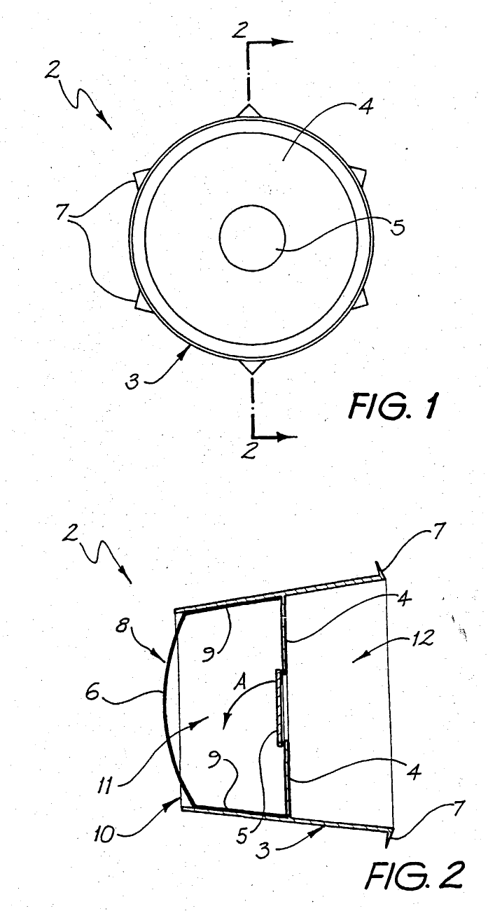

The occluding device shown in FIGS. 1 and 2 includes an elongate member in the form of a tapered tubular sleeve 3, a transverse partition 4 incorporating a flutter valve 5, and a frame 6. In this embodiment the periphery of the occluding device 2 is longitudinally tapered to aid insertion in a bronchus as described below, but this is not essential, as the tissue is normally sufficiently elastic to allow insertion.

The partition 4 subdivides the internal cavity of the occluding device 2 into a proximal rear section 11 and a distal head section 12. The flutter valve 5 is pivotally secured at one end to a wall portion of the partition 4 and moveable between an open and a closed position in the direction of Arrow A, FIG. 2. The flutter valve 5 is biased to the closed position, sealing the central aperture defined by the walls of the transverse partition 4 as shown in FIG. 2.

The head end of the sleeve 3 is provided with a series of equidistantly spaced peripheral projections 7. In use, each inclined projection 7 acts as a lateral anchor to prevent axial migration of the occluding device 2. Preferably the projections 7 are composed of a resilient material.

The frame 6 is coupled to the partition 4 and supports the rear sleeve section 11. The frame 6 essentially comprises an arcuate member 8 and an inwardly tapered skirt 9. A portion of the arcuate member 8 protrudes from mouth of the rear section 11 to act as a handle to assist in the insertion and/or removal of the occluding device 2.

The occluding device 2 can be utilised in a bronchoscopic procedure to selectively ‘sculpture’ the collapse of an emphysematous lung. The occluding device 2 is inserted and retained in the mouth of an endoscopic probe such that a portion of the distal section 12 protrudes from the mouth. Alternatively, the occluding device 2 can be grasped by the handle-like arcuate member 8. The probe is then introduced into the trachobronchial system by deployment through the nasal cavity, mouth/tracheal conduits of a patient. The probe is fed down the trachea into the bronchial tree of the target lung and positioned adjacent a pre-selected target site. For example, a third or fourth generation bronchus located in the apex of that lung.

The operator using visual and/or tactile feedback cues manipulates the occluding device 2 so that the occluding device becomes radially lodged in the bronchial cavity. If necessary, the arcuate member 8 is used as a handle for the probe to toggle the occluding device into position. The projections 7 engage or abut the bronchial wall of the target site and the rear section 11 is wedged like a cork, the elastic bronchial walls effecting an interference fit.

The probe is withdrawn from the patients body. Any tracheal or abdominal incision for insertion of bronchoscopic equipment is sutured appropriately,

The biased flutter valve 5 prevents the ingress of respiratory gas past the partition 4. The body will gradually absorb the gaseous content upstream of the occluding device 2. The blood flow to this lung section is minimised by the physiological hypoxic vasoconstriction. Occlusion of the bronchus by the occluding device 2 induces collapse of the downstream portion of the bronchial tree, functionally removing a section of the lung.

Fluid build-up is often associated with bronchial occlusion. In this case, the pressure of gas and mucous secretions adjacent the distal section 12 will override the bias of the flutter valve 5 allowing egress through the partition 4 and proximal section of the occluding device 2.

The occluding device 2 is removable by endoscopic probe retrieval. The frame 6 being coupled to the partition 4 enables radial collapse of the occluding device 2. The protruding portion of the arcuate member 8 is crushed and pulled downstream within the jaws of a probe to deform the skirt 9 and partition 4, compressing and dislodging the occluding device 2. The probe is withdrawn from the patients body.

It will be understood that the optimum location of the occluding device within the lung will be determined by the purpose of the intervention. As mentioned above, in the treatment of emphysema, a fourth generation bronchus may be preferred. In the treatment of pneumothorax, the location of the occluding device will be determined by the location of the breach in lung tissue. Where the device and method of the invention is used to isolate a diseased region of the lung, as in the treatment of tuberculosis, the clinician will determine the optimum location as part of the treatment strategy.

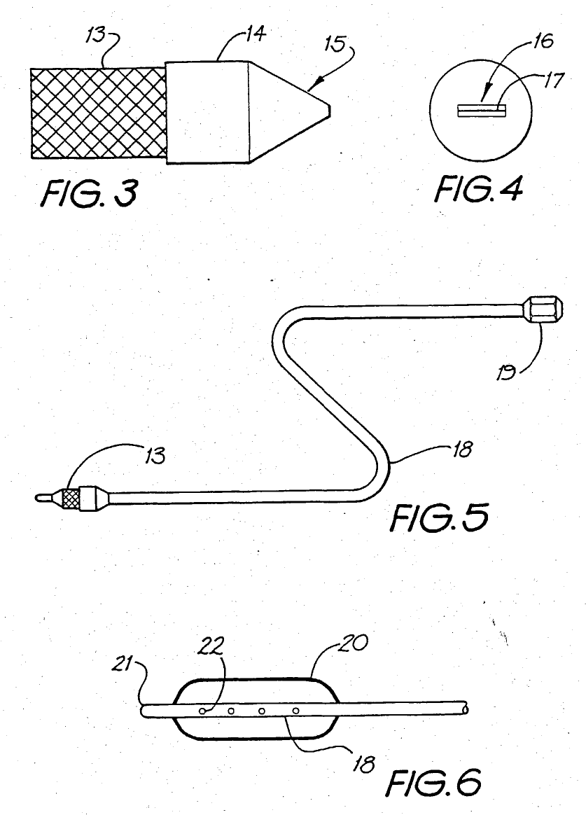

The occluding device shown in FIGS. 3 and 4 includes an expandable cylindrical stent 13, which may be of metal or plastics, carrying on its proximal end portion a valve member 14 which has a tapered end portion 15 forming a one-way valve having lips 16 and a slit 17. The valve member may be formed from a biologically compatible resilient plastics material such as silicone or polyurethane, or suitable biological materials. The device of FIGS. 3 and 4 is intended to be delivered by means of a system as illustrated in FIG. 5, consisting of a lumen 18 provided at its proximal end with a Luer connector 19 for attachment to an inflation device, and at its distal end with an inflatable and deflatable balloon 20, the lumen terminating in a rounded solid tip 21.

As shown in FIG. 6, the balloon 20 is sealed to the shaft of the lumen 18, and within the walls of the balloon 20 the shaft is provided with ports 22 for inflation and deflation of the balloon.

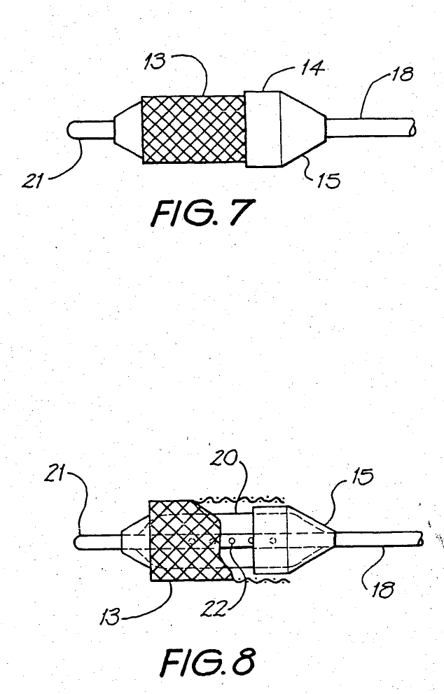

As shown in FIG. 7, the occluding device comprising the stent portion 13 and the valve 14 is mounted on the balloon 20 by passing the end of the lumen through the lips of the valve. Upon correct location of the device in the bronchus, the balloon 20 is inflated, expanding the stent portion and fixing the device in place against the bronchial wall. The stent portion 13 will normally be expanded to a diameter which is greater than the normal internal diameter of the bronchus at the site, so that upon relaxation after inflation the device remains in engagement with the bronchial wall. Sealing against the bronchial wall is provided by the material of the valve member 14.

In an alternative construction of such an occluding device, the valve member 14 may be fixed within, rather than outside, the stent body 13. Such an arrangement is shown in cross-section in FIG. 8. Where this arrangement is used, it may be preferred to attach the valve material to the stent device by suturing or glueing to achieve a gas-proof seal.

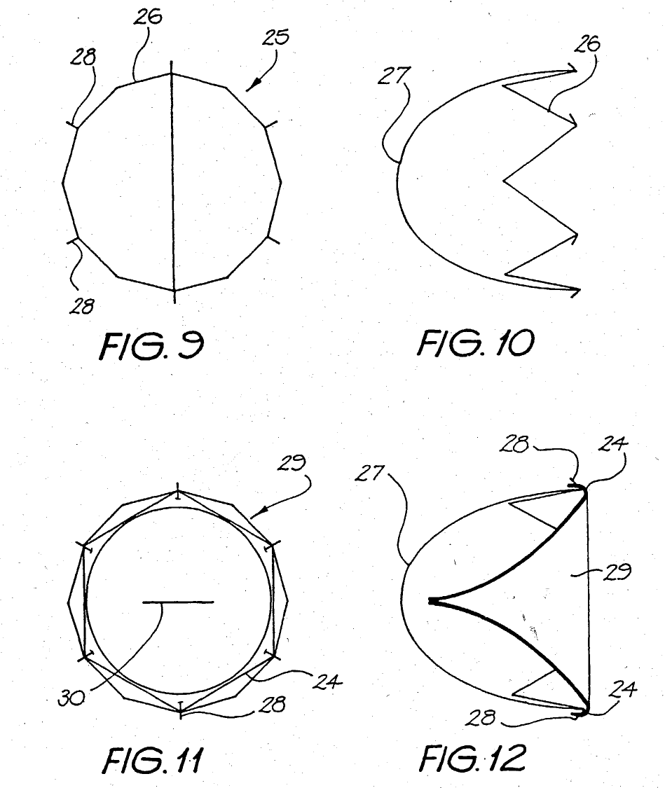

Another approach to the design of an occluding device for the purposes of the invention is shown in FIGS. 9 to 12. Here a frame 25 consisting of an expandable ring 26 and an arcuate “handle” 27 is also provided with barbs 28 around its periphery. A valve member of flexible material with a proximally directed valve aperture 30 is fixed within the frame 25 by having its outer edge 24 engaged over the barbs 28. Such a valve is capable of expanding into the position shown in FIGS. 11 and 12 with the frame 25, upon ejection from a delivery tube in which the device has been inserted, as described below.

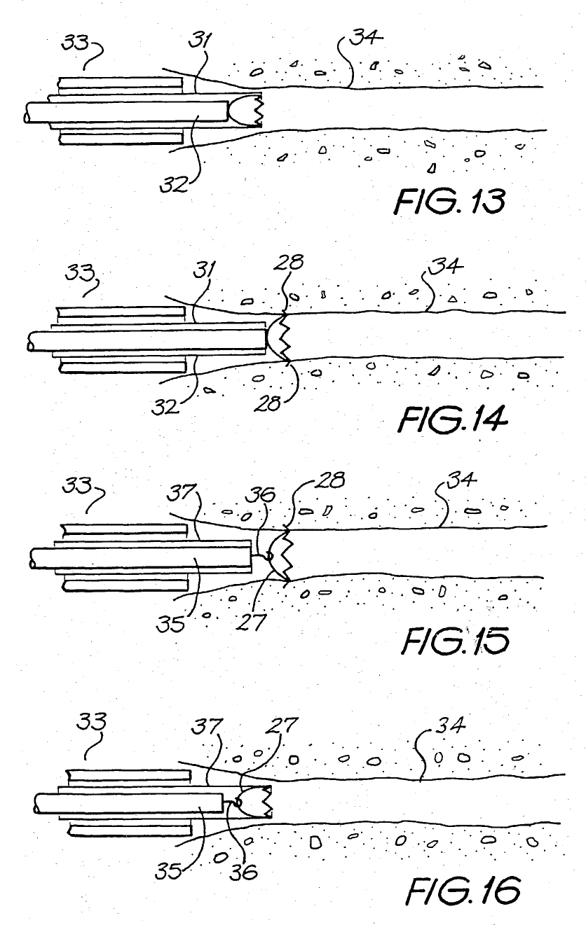

As shown in FIGS. 13 and 14, such a device may be located and fixed within the target bronchus 34 by means of a delivery tube 31 containing an ejector 32, mounted within the biopsy channel of a bronchoscope 33. The device is compressed within the delivery tube, and expands upon ejection, with the barbs 28 engaging the bronchial wall to resist migration of dislodgement of the device.

The frame 25 is preferably elastic so that it expands automatically into contact with the bronchial wall upon ejection, but alternatively it may be expanded by means of a balloon or other expanding device.

An advantage of the device of FIGS. 9-12 is that it is capable of removal by a simple endoscopic procedure. This is illustrated in FIGS. 15 and 16. A removal catheter consisting of an inner member 35 provided with a hook or grasping device 36 and an outer sheath 37 is deployed to the site by means of a bronchoscope 33. The hook 36 is engaged with the “handle” 27, and the sheath 37 advanced to compress the device, releasing the barbs 28 from the bronchial wall. The compressed device is then removed by withdrawing the members 35 and 37.

The devices and methods described above may be used in the treatment tuberculosis, particularly where multi-resistant strains are involved. In such a case the collapse of the target region of the lung following the introduction of an occluding device at the target site, and the subsequent hypoxic vasoconstriction, will rob bacilli in the target region of blood supply and effectively increase the potency of the antibiotics employed.

As mentioned above, the device may be manufactured from biodegradable material to remove the need for physical removal where persistence of the device is not required.

1. An endoscopic procedure for lung volumne reduction by inducing collapse of a target bronchial region including the steps of:
a) mounting an occluding device at the end of a probe,
b) positioning the occluding device by means of said probe at a predetermined target site in a bronchus accessing said target region
c) operating said probe so that the occluding device is lodged in the bronchus at said target site,
d) releasing the occluding device from said probe, and
e) retracting said probe
f) said occluding device being capable of sealing against the bronchial wall and including means substantially preventing the passage of gas or fluid to said target region.

2. A method of inducing lung volume reduction according to claim 1 including the further step of sealing said occluding device against the bronchial wall.

3. A method according to claim 2 wherein said sealing step includes the step of expanding said occluding device against the bronchial wall.

4. A method according to claim 3 wherein said expansion is achieved by inflating a balloon within said device.

5. A method according to claim 2 wherein said device is constrained within said probe and released therefrom at the target site to expand against the bronchial wall.

6. An endoscopic method for the treatment of pneumothorax including the steps of:
a) mounting an occluding device at the end of a probe,
b) positioning the occluding device by means of said probe at a predetermined target site in a bronchus accessing the affected region of the lung
c) operating said probe so that the occluding device is lodged in the bronchus at said target site,
d) releasing the occluding device from said probe, and
e) retracting said probe
f) said occluding device being capable of sealing against the bronchial wall and including means substantially preventing the passage of gas or fluid to said target region.

7. An endoseopic method for producing hypoxic vasoconstriction in a region of a lung as an adjunct to the chemotherapeutic treatment of lung disease, including the steps of:
a) mounting an occluding device at the end of a probe,
b) positioning the occluding device by means of said probe at a predetermined target site in a bronchus accessing said region
c) operating said probe so that the occluding device is lodged in the bronchus at said target site,
d) releasing the occluding device from said probe, and
e) retracting said probe
f) said occluding device being capable of sealing against the bronchial wall and including means substantially preventing the passage of gas or fluid to said target region.

8. The method of claim 7 in which the disease is tuberculosis.

9. A bronchiopulmonary occluding device including
a) a body adapted for sealing engagement with a bronchial wall, and
b) an obstruction substantially preventing flow of gas or fluid through said body in at least one direction.

10. A device according to claim 9 further including projections located on the periphery of the device and capable of engaging the bronchial wall upon location of the device at a target site.

11. A bronchiopulmonary occluding device according to claim 9 in which said obstruction includes a valve means allowing flow of gas or fluid through said body in one direction only.

12. A device according to claim 11 further including projections located on the periphery of the device and capable of engaging the bronchial wall upon location of the device at a target site.

13. A device according to claim 11 including an expandable hollow frame having attached thereto a one-way valve.

14. A device according to claim 13 further including projections located on the periphery of the device and capable of engaging the bronchial wall upon expansion of said frame.

15. A device according to claim 14 wherein said one-way valve is fixed to said frame by said projections.

16. A device according to claim 13 in which said frame is self-expandable upon ejection from a delivery device.

17. A device according to claim 16 including a member located proximally of said frame for gripping by an endoscopic device.

18. An endoscopic procedure for lung volume reduction by inducing collapse of a target bronchial region including the steps of:
a) mounting an occluding device in a delivery tube at the end of a probe,
b) said occluding device being capable of expanding upon ejection from said delivery tube,
c) positioning the occluding device by means of said probe at a predetermined target site in a bronchus accessing said target region
d) operating said probe so that the occluding device is ejected from said delivery tube at said target site, and
e) retracting said probe
f) said occluding device being capable of sealing against the bronchial wall and including means substantially preventing the passage of gas or fluid to said target region.

19. An endoscopic procedure for the removal of an occluding device from a bronchus including the steps of:
a) positioning a probe adjacent said occluding device
b) said probe including a capture tube and means for engaging said occluding device,
c) engaging said occluding device by said engaging means,
d) operating said probe so that the capture tube moves over said occluding device thereby collapsing said device and detaching it from engagement with the bronchial wall and
e) retracting said probe.

20. An endoscopic procedure for lung volume reduction by inducing collapse of a target bronchial region including the steps of:
a) mounting an occluding device at the end of a probe,
b) positioning the occluding device by means of said probe at a predetermined target site in a bronchus accessing said target region
(i) said occluding device being capable of sealing against the bronchial wall,
(ii) said occluding device including means substantially preventing the passage of gas or fluid to said target region,
(iii) said occluding device including means allowing the passage of gas or fluid from said target region,
c) operating said probe so that the occluding device is lodged in the bronchus at said target site,
d) releasing the occluding device from said probe, and
e) retracting said probe
f) thereby producing adsorption atelectasis of said target region.

21. An endoscopic procedure for lung volume reduction by inducing collapse of a target bronchial region including the steps of:
a) mounting an occluding device at the end of a probe,
b) positioning the occluding device by means of said probe at a predetermined target site in a bronchus accessing said target region
(i) said occluding device being capable of sealing against the bronchial wall,
(ii) said occluding device including means substantially preventing the passage of gas or fluid to said target region,
(iii) said occluding device including means allowing the passage of gas or fluid from said target region,
c) operating said probe so that the occluding device is lodged in the bronchus at said target site,
d) releasing the occluding device from said probe, and
e) retracting said probe
f) thereby producing adsorption atelectasis of said target region and hypoxic vasoconstriction therein.
